(19) United States
(12) Patent Application Publication
(10) Pub. No.: US 2009/0175935 A1
(43) Pub. Date: Jul. 9, 2009

(54) PHARMACEUTICAL COMPOSITIONS OF DULOXETINE

(75) Inventors: Umesh Setty, Gujarat (IN); Rakesh Kiritbhai Sheth, Gujarat (IN); Sujay Kamalakar Rajhans, Gujarat (IN); Pravin Meghraj Bhutada, Gujarat (IN); Hasmukh Mathurbhai Patel, Gujarat (IN)

Correspondence Address: PRICE HENEVELD COOPER DEWITT & LITTON, LLP, 695 KENMOOR, S.E., P O BOX 2567, GRAND RAPIDS, MI, 49501, US

(73) Assignee: TORRENT RESEARCH LTD., Gujarat (IN)

(21) Appl. No.: 11/993,283

(22) PCT Filed: Aug. 10, 2007
(86) PCT No.: PCT/IB07/02315

§ 371 Date: Feb. 20, 2009

(30) Foreign Application Priority Data
Aug. 14, 2006 (IN) 1279/MUM/2006

(51) Int. Cl. A61K 9/58; A61K 31/381; A61P 25/24; A61P 29/00; A61P 13/00

(52) U.S. Cl. 424/462; 514/438; 427/2.21

(57) ABSTRACT

The present invention relates to solid oral pharmaceutical compositions of duloxetine, process for preparing such compositions and method of using such compositions. Preferably, the invention relates to a delayed release composition of duloxetine comprising a core comprising duloxetine, optional separating coat and an enteric coat, wherein the enteric coat comprises methacrylic acid copolymer.

### TECHNICAL FIELD OF THE INVENTION

The present invention relates to solid oral pharmaceutical compositions of duloxetine, process for preparing such compositions and method of using such compositions. Preferably, the invention relates to a delayed release composition of duloxetine comprising a core comprising duloxetine, optional separating coat and an enteric coat, wherein the enteric coat comprises methacrylic acid copolymer.

### BACKGROUND OF THE INVENTION

Duloxetine is a mixed serotonin and norepinephrine reuptake inhibitor having a prominent antidepressant activity (Berk et al, Int Clin Psychopharmacol, 1997 May; 12(3):137-40). Chemically, duloxetine is designated as (+)-(S)-N-methyl-γ-(1-naphthyloxy)-2-thiophenepropylamine and is sold as its hydrochloride salt under the brand name Cymbalta® manufactured by Eli Lilly. U.S. Pat. No. 4,956,388 discloses the synthesis of duloxetine and its potent serotonin and norepinephrine uptake inhibitory property.

U.S. Pat. No. 5,508,276 assigned to Elli Lilly and Co. discloses that it is advisable to formulate duloxetine in an enteric form due to its instability in acidic solutions. It also teaches enteric formulation of duloxetine in the form of enteric pellets of which the enteric coat comprises hydroxypropyl methylcellulose acetate succinate (HPMCAS). The selection of HPMCAS as the polymer was arrived after finding that duloxetine reacted with many enteric coatings to form a slowly or even insoluble coating. It is disclosed that the use of a polymer other than HPMCAS formed a pellet formulation having a disadvantageous drug-release profile and low bioavailability. Jansen et al, J Pharm Sci, 87 (1), 1998: 81-85 discloses that duloxetine reacts with degradation products or residual free acids present in the enteric polymer such as hydroxypropyl methylcellulose phthalate to form impurities such as phthalamide impurities. U.S. Pat. No. 5,508,276 also describes the difficulty of preparing high-loaded enteric formulation of duloxetine which would not release duloxetine in acid environments. The marketed formulation of Cymbalta® comprises enteric pellets as taught by U.S. Pat. No. 5,508,276.

U.S. Pat. No. 5,910,319 assigned to Eli Lilly discloses enteric fluoxetine pellet comprising a) a core consisting of fluoxetine and one or more pharmaceutically acceptable excipients; b) an optional separating layer comprising a non-reducing sugar and one or more pharmaceutically acceptable excipients; c) an enteric layer comprising hydroxypropylmethylcellulose acetate succinate (HPMCAS) and one or more pharmaceutically acceptable excipients; d) an optional finishing layer. The said compositions were described to provide a convenient and effective once per week dosing of higher doses of fluoxetine (e.g., 60-120 mg), having blunt initial release of fluoxetine and lesser side effects.

PCT Application No. 2003/13480 filed by Dr. Reddy's Laboratories Ltd. discloses an enteric fluoxetine formulation comprising: (a) a core comprising fluoxetine or a pharmaceutically accepted salt, solvate, enantiomers or mixtures thereof including racemic mixture, in an amount of 90 mg base equivalent of fluoxetine, (a) an optional smoothening layer, (a) an enteric coating layer comprising an at least one enteric coating polymers selected from the group consisting of Eudragit L100-55, Eudragit L 100, Eudragit S 100, hydroxypropyl methylcellulose pthalate, cellulose acetate pthalate, polyvinyl acetate pthalate; an at least one plasticisers selected from the group consisting of triethyl citrate, polyethylene glycol, diethyl pthalate or dibutyl pthalate; an at least one lubricant or glidants selected from the group consisting of talc, magnesium stearate, kaolin or colloidal silicon dioxide, and (a) an optional finishing layer. The composition was described to be advantageous as it could be prepared without a separating coat. The composition was found to be therapeutically equivalent to the commercially available product Prozac® Weekly 90 mg capsule.

US 2006/165776 patent application discloses an oral pharmaceutical composition of duloxetine comprising a core comprised of an inert nuclei and duloxetine mixed and compressed together, an intermediate layer and an enteric layer, wherein the composition is free of alkaline reacting compounds. It also discloses the compositions and methods for preparing micro-tablets of duloxetine.

There is still a need in the art to prepare enteric formulation of duloxetine which are stable with respect to impurities and degradation products and would have maximum release of duloxetine in the intestine. It was surprisingly found that enteric formulation of duloxetine can be prepared with methacrylic acid copolymer in the enteric coat, without compromising the drug-release and bioavailability.

### SUMMARY OF THE INVENTION

One aspect discloses a delayed release pharmaceutical composition comprising:
(i) a core comprising an inert core coated with duloxetine;
(ii) optionally a separating coat on the core; and
(iii) an enteric coat on the core or on the separating coat,
   wherein the enteric coat comprises methacrylic acid copolymer.

Another aspect discloses a process for preparation of a delayed release pharmaceutical composition comprising:
(i) preparing an inert core;
(ii) coating the inert core with duloxetine;
(iii) optionally coating the product of step (ii) with a separating coat; and
(iv) coating the product of step (ii) or (iii) with an enteric coat,
   wherein the enteric coat comprises methacrylic acid copolymer.

Yet another aspect discloses a method for treatment of major depressive disorder, management of diabetic neuropathic pain associated with diabetic peripheral neuropathy, treatment of moderate to severe stress urinary incontinence in women, comprising administering to a patient in need thereof a delayed release pharmaceutical composition comprising:
(i) a core comprising an inert core coated with duloxetine;
(ii) optionally a separating coat on the core; and
(iii) an enteric coat on the core or on the separating coat,
   wherein the enteric coat comprises methacrylic acid copolymer.

### DETAILED DESCRIPTION OF THE INVENTION

The term “delayed release pharmaceutical composition” as described herein is intended to include compositions which provide a maximum release of duloxetine in the less acidic environment of the intestine relative to the more acidic environment of the stomach.

The term “duloxetine” as described herein is intended to include duloxetine free base or pharmaceutically acceptable acid addition salts thereof, racemic mixture, individual enantiomer or mixtures thereof. The preferred salt is duloxetine hydrochloride. The particle size of duloxetine as used herein may vary from 1 μm to 200 μm.

The term “core” as described herein is intended to include anything below the separating coat or when the separating coat is absent, anything below the enteric coat. Thus the core may contain inert core covered with duloxetine, core containing duloxetine, or mixtures thereof. The inert core may comprise inert non-pareils which are conventionally used in pharmaceutical industry and are readily available. The inert non-pareils may be of any pharmaceutically acceptable excipient such as starch, sugar, microcrystalline cellulose, vegetable gums, waxes, and the like. Preferably, the inert non-pareils are of starch and sugar. The size of the inert non-pareils may vary from 0.1 mm-2 mm. The core may also be prepared by techniques such as granulation or extrusion-spheronization. For example, the core may be prepared by mixing one or more pharmaceutically acceptable excipient and duloxetine, moistening the mixture with water or a solvent, granulating and subsequently drying to obtain granules which may be used as the core. Alternatively, such granules may be compressed into a tablet, which may be used as the core. The core may also be prepared by mixing one or more pharmaceutically acceptable excipient and duloxetine, wetting with water or organic solvent and mixing in a high shear granulator to form a homogeneous wet mass, extruding the wet mass to form extrudates which are subsequently spheronized to form spheres which may be used as the core. The core may be present in an amount ranging from 10 % to 90 % by weight of the composition.

The delayed release compositions may comprise a separating coat between the core and the enteric coat. The separating coat may provide stability by inhibiting direct contact of the components of the core and the enteric polymer in the enteric coat. The separating coat may also provide protection to the core during its passage from the stomach to the intestines. Preferably, the separating coat is compatible with duloxetine and the enteric coat and does not affect the dissolution of the composition. The separating coat may comprise one or more film forming polymer such as ethyl cellulose, hydroxypropyl cellulose, hydroxypropyl methylcellulose, polyvinyl pyrrolidone; and the like. The separating coat may be prepared by dissolving an appropriate amount of film forming polymer into a suitable solvent system such as water, organic solvent such as alcohol, methylene chloride, and the like; or mixtures thereof, and spraying the solution or suspension on core using a suitable apparatus. The separating coat may be present in an amount ranging from 0.5% to 30% by weight of the composition.

The “enteric coat” as described herein may comprise a suitable pH-dependent polymer selected from cellulose acetate phthalate, cellulose acetate succinate, methylcellulose phthalate, hydroxypropyl methylcellulose phthalate, ethylhydroxycellulose phthalate, polyvinylacetate phthalate, polyvinyl butyrate acetate, vinyl acetate-maleic anhydride copolymer, styrene-maleic mono-ester copolymer, polymethacrylates such as methyl acrylate-methacrylic acid copolymer, methacrylate-methacrylic acid-octyl acrylate copolymer, hydrogenated castor oil, and the like. The polymer may be used either alone or in combination with other polymers. Preferably, the enteric polymer may be selected from the various pharmaceutically acceptable polymethacrylates, preferably methacrylic acid co-polymers, more preferably co-polymers based on methacrylic acid and methyl methacrylate sold under the brand name EUDRAGIT®. Examples include EUDRAGIT® L series (a cationic polymer synthesized from dimethylaminoethyl methacrylate) such as EUDRAGIT® L 12.5, EUDKAGIT® L 12.5P, EUDRAGIT® L 100, EUDRAGIT® L 100-55, EUDRAGIT® L-30, EUDRAGIT® L-30 D-55; the EUDRAGIT® S series such as EUDRAGIT® S 12.5, EUDKAGIT® S 12.5P, EUDRAGIT® S100; the EUDRAGIT® NE series such as EUDRAGIT® NE 30D; the EUDRAGIT® RL series such as EUDRAGIT® RL 12.5, EUDRAGIT® RL 100, EUDKAGIT® RL PO, EUDRAGIT® RL 30D; and the EUDRAGIT® RS series such as EUDRAGIT® RS 12.5, EUDKAGIT® RS 100, EUDRAGIT® RS PO, EUDRAGIT® RS 30D; and the like. The enteric polymer, such as methacrylic acid copolymer, may be neutralized to an appropriate pH by using alkaline substances, such as sodium hydroxide, potassium hydroxide or ammonium hydroxide; and the like, and the neutralized polymer may be used in preparation of the enteric coat. The enteric coat may be applied by dispersing or suspending the enteric polymer in a suitable medium, such as water or aqueous acidic or alkaline solutions, or in organic solvents such as methanol, ethanol, isopropanol, acetone, methyl ethyl ketone, methylene chloride, ethylene chloride, ethyl acetate, or mixtures thereof, and the resultant solution or suspension may be sprayed directly on the core or separating coat, followed by drying to obtain delayed release composition. The enteric coat may be present in an amount ranging from 5% to 60% by weight of the composition. The enteric coating polymer may be present in an amount ranging from 5-50% by weight, more preferably 10-30% by weight of the composition.

The pharmaceutical compositions as described herein may additionally comprise one or more pharmaceutically acceptable excipients selected from diluent, disintegrant, binder, lubricant, glidant, plasticizer, anti-tacking agent, opacifying agent, and the like.

Diluent may be selected from powdered cellulose, microcrystalline cellulose, silicified microcrystalline cellulose, lactose, starch, dibasic calcium phosphate, tribasic calcium phosphate, calcium carbonate, dextrates, dextrin, dextrose, kaolin, magnesium carbonate, magnesium oxide, sugars such as sucrose; sugar alcohols such as mannitol, sorbitol, erythritol; and mixtures thereof. Diluent may generally be added to increase the bulk volume of the powder to facilitate granulation or compression. Diluent, such as a sugar, may also be added as a component of the coat, such as in the separating coat, to impart sticking and acid-resistance properties to the coating layer. The diluent may be present in an amount ranging from 1% to 80% by weight of the composition.

Disintegrant may be selected from croscarmellose sodium, sodium starch glycolate, pregelatinized starch, sodium carboxymethyl cellulose, microcrystalline cellulose, cross-linked polyvinylpyrrolidone, sodium alginate and mixtures thereof. The disintegrant may be present in an amount ranging from 1% to 20% by weight of the composition.

Binder may be selected from hydroxyethyl cellulose, hydroxypropyl cellulose, hydroxypropyl methylcellulose, carbomers, carboxymethylcellulose sodium, dextrin, ethyl cellulose, methylcellulose, shellac, zein, gelatin, polymethacrylates, polyvinyl pyrrolidone, pregelatinized starch, sodium alginate, gums, synthetic resins and the like. Binder may be used as a component of the coat to ensure proper adhesion of the subsequent coats. The binder may be present in an amount ranging from 0.1% to 25% by weight of the composition.

Lubricant may be selected from metallic stearates such as magnesium stearate, calcium stearate, zinc stearate; stearic acid, hydrogenated vegetable oil, hydrogenated castor oil, glyceryl palmitostearate, glyceryl behenate, polyethylene glycols, corn starch, sodium stearyl fumarate, sodium benzoate, mineral oil, talc, and mixtures thereof. Glidant may be selected from talc, colloidal silicon dioxide, magnesium trisilicate, powdered cellulose, starch, tribasic calcium phosphate; and mixtures thereof. The lubricant or glidant may be present in an amount ranging from 0.1% to 10% by weight of the composition.

Plasticizer may be used in a coat to increase the flexibility and strength of the layer and may be selected from propylene glycol, polyethylene glycol, triethyl citrate, acetyl triethyl citrate, diethyl phthalate, dibutyl phthalate, dibutyl sebacate; or mixtures thereof. The plasticizer may be present in an amount ranging from 0.1% to 20% by weight of the composition.

Anti-tacking agent may be used in a coat to aid bulk build-up and form a smooth surface and may be selected from talc, kaolin, finely divided silicon dioxide, glyceryl monostearate, and the like. The anti-tacking agent may be present in an amount ranging from 0.1% to 20% by weight of the composition.

Opacifying agent may be used in a coat to prevent photo-degradation and may be selected from titanium dioxide, iron oxides, and the like. The opacifying agent may be present in an amount ranging from 0.1% to 10% by weight of the composition.

The pharmaceutical compositions as described herein may be prepared by different techniques. For example, when an inert core is used, non-pareils may be coated with a seal coat comprising a film forming polymer, e.g. ethylcellulose, and excipients like plasticizer, anti-tacking agent and opacifying agent. The components of the seal coat may be dissolved or dispersed in an appropriate solvent and the dispersion may be coated on the core in a conventional coating pan or fluidized bed equipment (such as a Wurster or Glatt) and the coated cores may then be dried. A coat of duloxetine may then be applied to such coated cores using similar process as above, wherein duloxetine may be built up on the coated cores by spraying a suspension or dispersion comprising duloxetine and excipients such as binder, plasticizer, anti-tacking agent and opacifying agent. Alternatively, the duloxetine coat may also be applied by powder-coating, wherein the coated cores as described above are maintained in a sticky state, a mixture of duloxetine and powdered excipients such as binder, plasticizer, anti-tacking agent and opacifying agent are added continuously or periodically so as to adhere to the sticky cores. When the entire duloxetine coat has been applied, the drug coated cores are dried. The drug-coated cores may optionally be coated with a separating coat or may directly be coated with the enteric coat. The enteric coat may be applied by dispersing or suspending the enteric polymer in a suitable medium which may additionally comprise excipients such as plasticizer, anti-tacking agent and opacifying agent, and the resultant dispersion may be sprayed on the drug-coated cores, followed by drying to obtain enteric-coated pellets. The enteric pellets may be filled into capsules of suitable size or provided as any suitable composition such as tablet or sachet.

The compositions may also be prepared by providing a core prepared by techniques such as granulation. For example, pharmaceutically acceptable excipients such as diluent, disintegrant, binder, glidant, and duloxetine may be mixed; the mixture may be moistened with water or a solvent, granulated and subsequently dried to obtain granules which may be used as the core. The core may be optionally coated with a separating coat or directly coated with the enteric coat by processes as described herein to obtain pellets which may be filled into capsules of suitable size or provided as any suitable composition such as tablet or sachet. Alternatively, the uncoated granules may be lubricated and compressed into a tablet, which may be used as the core. The tablet may be optionally coated with a separating coat and subsequently coated with the enteric coat.

The compositions may also be prepared by providing a core prepared by techniques such as extrusion-spheronization wherein one or more pharmaceutically acceptable excipient and optionally duloxetine are mixed and wetted with water or organic solvent in a high shear granulator to form a homogeneous wet mass, the wet mass is extruded to form extrudates which are subsequently spheronized to form spheres, which may be used as the core. The core may be optionally coated with a separating coat and subsequently coated with the enteric coat by processes as described herein to obtain pellets which may be filled into capsules of suitable size or provided as any suitable composition such as tablet or sachet. Alternatively, the uncoated cores may be compressed into a tablet, which may be used as the core. The tablet may be optionally coated with a separating coat and subsequently coated with the enteric coat.

In one embodiment, delayed release duloxetine compositions may be prepared by
providing inert non-pareils;
coating the inert non-pareils with duloxetine;
coating the drug-coated cores with a separating coat;
coating the product obtained above with an enteric coat;
optionally mixing the enteric-coated pellets with one or more pharmaceutically acceptable excipient; and
filling the enteric-coated pellets into capsules.

In another embodiment, delayed release duloxetine compositions may be prepared by
preparing a core by mixing duloxetine and one or more pharmaceutically acceptable excipient selected from the group consisting of diluent, disintegrant, glidant and binder;
granulating the mixture with a solvent or a binder solution,
drying the granules;
optionally coating the granules with a separating coat;
coating the product obtained above with an enteric coat;
optionally mixing the enteric-coated granules with one or more pharmaceutically acceptable excipient; and
filling the enteric-coated granules into capsules or compressing into tablets.

In another embodiment, delayed release duloxetine compositions may be prepared by
preparing a core by mixing one or more pharmaceutically acceptable excipient selected from the group consisting of diluent, disintegrant, glidant and binder;
granulating the mixture with a solvent,
drying the granules;
coating the granules with duloxetine;
optionally coating the granules with a separating coat;
coating the product obtained above with an enteric coat
optionally mixing the enteric-coated granules with one or more pharmaceutically acceptable excipient; and
filling the enteric-coated granules into capsules or compressing into tablets.

The pharmaceutical compositions as described herein may be illustrated by the following example which is not to be construed as limiting the scope of the invention:

### Example 1

### Capsules of Enteric Duloxetine Pellets Comprising Methacrylic Acid/Methacrylate Co-Polymer in the Enteric Coat

| | | Quantity |
|---|---|---|
| S/N | Ingredients | (% of comp.) |
| 1 | Nonpareils | 20-60% |
| 2 | Ethyl cellulose | 2-6% |
| 3 | Talc | 5-15% |
| 4 | Duloxetine HCl | 1-99% |
| 5 | Hydroxypropyl methylcellulose | 1-15% |
| 6 | Colloidal silicon dioxide | 1-5% |
| 7 | Methacrylic acid/methacrylate copolymer | 5-20% |
| 8 | Titanium dioxide | 0.1-2% |
| 9 | Triethyl citrate | 1-5% |
| 10 | Methanol/methylene chloride or purified water | q.s. |

PROCEDURE: Non-pareils were sifted and passed through a sieve of appropriate size. Ethylcellulose and talc were dispersed in water and the dispersion was sprayed on non-pareils in a fluid bed apparatus. Duloxetine, hydroxypropyl methylcellulose and colloidal silicon dioxide were dissolved in water or dispersed in a mixture of methylene chloride/methanol and the solution or dispersion was sprayed on the ethylcellulose coated non-pareils. The drug-coated cores were coated with a separating coat by spraying a solution of hydroxypropyl methylcellulose in a mixture of methylene chloride/methanol. Methacrylic acid/methacrylate co-polymer, titanium dioxide and triethyl citrate were dispersed in water or aqueous solution of methanol (depending on the solubility of methacrylic-acid copolymer) and the dispersion was sprayed on the drug-coated non-pareils to obtain enteric pellets, which were filled in a capsule of suitable size.

### Example 2

| | | Quantity |
|---|---|---|
| S/N | Ingredients | (mg/capsule) |
| 1 | Sugar spheres | 119.16 |
| 2 | Ethyl cellulose | 5.23 |
| 3 | Talc | 17.21 |
| 4 | Duloxetine hydrochloride | 67.40 |
| 5 | Hydroxypropyl methylcellulose (HPMC) | 42.60 |
| 6 | Methacrylic acid/methacrylate copolymer | 80.80 |
| | (Eudragit L30D55) | |
| 7 | Triethyl citrate | 8.07 |
| 8 | Methanol | q.s. |
| 9 | Methylene chloride | q.s. |
| 10 | Purified water | q.s. |
| | Total | 340.47 |

PROCEDURE: Sugar spheres were sifted and passed through a sieve of appropriate size. Ethylcellulose and talc were dispersed in a mixture of methanol and methylene chloride and the dispersion was sprayed on sugar spheres. Duloxetine was suspended in hydroxypropyl methylcellulose solution in purified water and dispersion was sprayed on the seal-coated sugar spheres. The drug-coated pellets were coated by spraying a solution of hydroxypropyl methylcellulose in purified water. Methacrylic acid/methacrylate co-polymer (Eudragit L30D 55), triethyl citrate and talc were dispersed in purified water and the dispersion was sprayed on the HPMC-coated pellets to obtain enteric pellets, which were filled in a capsule of suitable size.

### Example 3

| | | Quantity |
|---|---|---|
| S/N | Ingredients | (mg/capsule) |
| 1 | Sugar spheres | 150.00 |
| 2 | Talc | 30.39 |
| 3 | Duloxetine hydrochloride | 67.20 |
| 4 | Hydroxypropyl methylcellulose (HPMC) | 26.67 |
| 5 | Sucrose | 4.74 |
| 6 | Methacrylic acid/methacrylate copolymer | 69.84 |
| | (Eudragit L30D55) | |
| 7 | Triethyl citrate | 13.95 |
| 8 | Sodium hydroxide | 0.54 |
| 9 | Polyethylene glycol (PEG 400) | 0.12 |
| 10 | Titanium dioxide | 0.78 |
| 11 | Purified water | q.s. |
| | Total | 364.23 |

PROCEDURE: Sugar spheres were sifted and passed through a sieve of appropriate size. Hydroxypropyl methylcellulose and talc were dispersed in purified water and the dispersion was sprayed on sugar spheres. Duloxetine was suspended in hydroxypropyl methylcellulose solution in purified water and dispersion was sprayed on the seal-coated sugar spheres. The drug-coated pellets were coated by spraying a solution of sucrose and hydroxypropyl methylcellulose in purified water. Methacrylic acid/methacrylate co-polymer (Eudragit L30D 55) and triethyl citrate were dispersed in purified water and the dispersion was neutralized with 0.1 N sodium hydroxide solution up to pH 5.5. Talc was added to the neutralized dispersion and the dispersion was sprayed on the HPMC-coated pellets to obtain enteric pellets. The enteric coated pellets were then coated with a dispersion of hydroxypropyl methylcellulose, polyethylene glycol (PEG 400), titanium dioxide and talc in purified water, which were filled in a capsule of suitable size.

### Example 4

| | | Quantity |
|---|---|---|
| S/N | Ingredients | (mg/capsule) |
| 1 | Sugar spheres | 180 |
| 2 | Ethyl cellulose | 10.8 |
| 3 | Talc | 38.4 |
| 4 | Duloxetine hydrochloride | 67.2 |
| 5 | Hydroxypropyl methylcellulose (HPMC) | 22.42 |
| 6 | Methacrylic acid/methacrylate copolymer | 108.67 |
| | (Eudragit L30D55) | |
| 7 | Triethyl citrate | 10.87 |
| 8 | Methanol | q.s. |
| 9 | Methylene chloride | q.s. |
| 10 | Purified water | q.s. |
| | Total | 438.36 |

PROCEDURE: Sugar spheres were sifted and passed through a sieve of appropriate size. Ethylcellulose and talc were dispersed in a mixture of methanol and methylene chloride and the dispersion was sprayed on sugar spheres. Duloxetine was suspended in hydroxypropyl methylcellulose solution in purified water and dispersion was sprayed on the seal-coated sugar spheres. The drug-coated pellets were coated by spraying a solution of hydroxypropyl methylcellulose in purified water. Methacrylic acid/methacrylate co-polymer (Eudragit L30D 55), triethyl citrate and talc were dispersed in purified water and the dispersion was sprayed on the HPMC-coated pellets to obtain enteric pellets, which were filled in a capsule of suitable size.

TABLE 1
Comparative dissolution profiles of Example 3 and Reference
product in USP type I dissolution apparatus in 0.1 N HCl for
first 2 hours followed by phosphate buffer having pH 6.8.

% drug dissolved

| | | Reference |
|---|---|---|
| Time | Example 3 | product* |
| 2 hrs | 0.2 | 0.1 |

6.8 phosphate buffer

| | | |
|---|---|---|
| 15 min | 55.7 | 53.7 |
| 30 min | 88.6 | 78.6 |
| 45 min | 92.6 | 92.4 |
| 60 min | 93.9 | 95.8 |

*Reference product refers to Duloxetine Enteric Capsules (60 mg), sold under the brand name Cymbalta ® by Eli-Lilly in USA

As is evident from Table 1, the compositions as described herein exhibit dissolution comparable to the Reference product.

We claim:

1. A delayed release pharmaceutical composition comprising:
(i) a core comprising an inert core coated with duloxetine;
(ii) optionally a separating coat on the core; and
(iii) an enteric coat on the core or on the separating coat,
wherein the enteric coat comprises methacrylic acid copolymer.

2. The composition of claim 1, wherein the methacrylic acid copolymer is present in an amount ranging from 10 to 30% by weight of the composition.

3. The composition of claim 1, wherein the core is present in an amount ranging from 10 to 90% by weight of the composition.

4. The composition of claim 1, wherein the separating coat is present in an amount ranging from 0.5 to 30% by weight of the composition.

5. The composition of claim 1, wherein the composition further comprises one or more pharmaceutically acceptable excipients selected from diluent, disintegrant, binder, lubricant, glidant, plasticizer, anti-tacking agent or opacifying agent.

6. The composition of claim 5, wherein the diluent is selected from the group consisting of powdered cellulose, microcrystalline cellulose, silicified microcrystalline cellulose, lactose, starch, dibasic calcium phosphate, tribasic calcium phosphate, calcium carbonate, dextrates, dextrin, dextrose, kaolin, magnesium carbonate, magnesium oxide, sucrose, mannitol, sorbitol, erythritol, and mixtures thereof.

7. The composition of claim 5, wherein the disintegrant is selected from the group consisting of croscarmellose sodium, sodium starch glycolate, pregelatinized starch, sodium carboxymethyl cellulose, microcrystalline cellulose, cross-linked polyvinylpyrrolidone, sodium alginate and mixtures thereof.

8. The composition of claim 5, wherein the binder is selected from the group consisting of hydroxyethyl cellulose, hydroxypropyl cellulose, hydroxypropyl methylcellulose, carbomer, carboxymethylcellulose sodium, dextrin, ethyl cellulose, methylcellulose, shellac, zein, gelatin, polymethacrylates, polyvinyl pyrrolidone, pregelatinized starch, sodium alginate, gums, synthetic resins and mixtures thereof.

9. The composition of claim 5, wherein the lubricant is selected from the group consisting of magnesium stearate, calcium stearate, zinc stearate, stearic acid, hydrogenated vegetable oil, hydrogenated castor oil, glyceryl palmitostearate, glyceryl behenate, polyethylene glycols, sodium stearyl fumarate, sodium benzoate, mineral oil, talc and mixtures thereof.

10. The composition of claim 1, wherein the composition is a pellet or a capsule.

11. A process for preparation of a delayed release pharmaceutical composition comprising:
(i) preparing an inert core;
(ii) coating the inert core with duloxetine;
(iii) optionally coating the product of step (ii) with a separating coat; and
(iv) coating the product of step (ii) or (iii) with an enteric coat,
wherein the enteric coat comprises methacrylic acid copolymer.

12. A method for treatment of major depressive disorder, management of diabetic neuropathic pain associated with diabetic peripheral neuropathy, treatment of moderate to severe stress urinary incontinence in women, comprising administering to a patient in need thereof a delayed release pharmaceutical composition comprising:
(i) a core comprising an inert core coated with duloxetine;
(ii) optionally a separating coat on the core; and
(iii) an enteric coat on the core or on the separating coat,
wherein the enteric coat comprises methacrylic acid copolymer.
